(19) United States
(12) Patent Application Publication
(10) Pub. No.: US 2021/0115437 A1
(43) Pub. Date: Apr. 22, 2021

(54) IN VITRO SYNTHESIS METHOD FOR SGRNA AND KIT THEREOF

(71) Applicant: SHANGHAI SINOBIO BIOTECH CO. LTD., Shanghai (CN)

(72) Inventors: Huaxing ZHU, Shanghai (CN); Qingyi ZHANG, Shanghai (CN); Jiajia SHI, Shanghai (CN); Manman ZHAO, Shanghai (CN); Yi HE, Shanghai (CN)

(21) Appl. No.: 17/043,119

(22) PCT Filed: Mar. 29, 2019
(86) PCT No.: PCT/CN2019/080485

§ 371 Date: Jan. 4, 2021

(30) Foreign Application Priority Data
Mar. 29, 2018 (CN) 201810271411.2

(51) Int. Cl. C12N 15/11; C12N 9/22; C12P 19/34

(52) U.S. Cl. CPC C12N 15/11; C12N 9/22; C12N 2800/80; C12N 2310/20; C12P 19/34

(57) ABSTRACT

The present invention provides an in vitro synthesis method for an sgRNA and a kit thereof. Specifically, the present invention provides a nucleic acid construct having a structure represented by formula I from 5′ to 3′: Y1-L1-Y2-L2-Y3-Y4 (I), wherein Y1 is an RNA polymerase promoter region; L1 is absent or a linking sequence; Y2 is a target DNA sequence; L2 is absent or a linking sequence; Y3 is a downstream primer binding region; Y4 is absent or a nucleotide sequence; and each “-” is independently a bond or a nucleotide linking sequence.

### INCORPORATION OF SEQUENCE LISTING

This application contains a sequence listing in Computer Readable Form (CRF). The CFR file containing the sequence listing entitled “PB4084183-SequenceListing.txt”, which was created on Dec. 29, 2020, and is 1,510 bytes in size. The information in the sequence listing is incorporated herein by reference in entirety.

### TECHNICAL FIELD

The present invention relates to the field of biotechnology, in particular to an in vitro synthesis method of sgRNA and a kit thereof.

### BACKGROUND

The CRISPR (clustered regularly interspaced short palin-dromic repeats)/Cas (CRISPR-associated) system is a unique acquired immune system in some bacteria and archaebacteria. This system is guided by a specific sequence of RNA to specifically cut and degrade exogenous DNA. The CRISPR/Cas system can be divided into three types, in which the type II CRISPR/Cas system has been transformed into a tool for targeted genome editing due to its simple composition. By artificially designing and transcribing RNA in vitro, sgRNA (single guide RNA) with guiding effect can be synthesized, and the sgRNA guides Cas protein to specifically cut the target DNA sequence. Through the modification of Cas protein, under the guidance of sgRNA, the CRISPR/Cas system can achieve a variety of purposes in research, such as cutting, modifying, silencing, knocking out, and adjusting expression of genes. CRISPR/Cas has become a powerful tool for gene editing and research, and has a very bright and broad application prospect.

As an indispensable sgRNA in the CRISPR/Cas system, its synthesis method is very important. How to obtain available sgRNA at a lower cost has become the focus of scientific researchers. The time period of chemical synthesis of RNA is 7 to 14 days, and the cost is very high, which is not suitable for sgRNA synthesis. The method of constructing plasmid vectors to transcribe RNA in vivo requires tedious and time-consuming vector construction work, and is not flexible enough. Each vector can only correspond to a small amount of specific RNA.

At present, the traditional sgRNA in vitro transcription method has many tedious steps, usually, 1. the template DNA is amplified by PCR, 2. the PCR product is recovered through gels, and 3. sgRNA is transcribed using the PCR product as a template. The operation is time-consuming and laborious, and the entire process takes 5-6 hours. A PCR machine, an electrophoresis tank, a centrifugal machine and other instruments are needed during the process. The reagents involved include PCR amplification enzyme, gel recovery kit, T₇ RNase, etc., so that the average cost is not low.

Therefore, there is an urgent need in the art to develop a simple, convenient, time-saving, efficient and economical sgRNA in vitro synthesis method.

### SUMMARY OF THE INVENTION

The present invention provides a simple, convenient, time-saving, efficient and economical sgRNA in vitro synthesis method.

In a first aspect of the present invention, it provides a sgRNA synthesis system, comprising:

(a) a nucleic acid construct, which has a 5′-3′ structure as shown in Formula I:

Y1-L1-Y2-L2-Y3-Y4 (I)

wherein

Y1 is a RNA polymerase initiation region;

L1 is none or a linker sequence;

Y2 is a target DNA sequence;

L2 is none or a linker sequence;

Y3 is a reverse primer binding region;

Y4 is none or a nucleotide sequence;

and, each “-” is independently a bond or a nucleotide linker sequence;

(b) a reverse primer;

(c) a DNA polymerase; and

(d) a RNA polymerase.

In another preferred embodiment, the RNA polymerase is selected from the group consisting of T7 RNA polymerase, Sp6 RNA polymerase, U6 RNA polymerase, T3 RNA polymerase, and a combination thereof.

In another preferred embodiment, the RNA polymerase initiation region is a T7 RNA polymerase initiation region.

In another preferred embodiment, the sequence structure of the element Y1 is: N_{X}-TAATACGACTCACTATA (positions 2-18 of SEQ ID NO.:1)-G_{Y}, wherein N is A, T, C or G, and X is an integer of 1-6, Y is an integer of 0-2.

In another preferred embodiment, the sequence of the element Y1 is shown in SEQ ID NO.:1.

In another preferred embodiment, the sequence of the element Y1 is shown in SEQ ID NO.:2.

In another preferred embodiment, the sequence of the element Y1 is shown in SEQ ID NO.:3.

In another preferred embodiment, the length of the element Y1 is 17-40 bp, preferably 18-25 bp.

In another preferred embodiment, the length of the element Y2 is 0-100 bp, preferably, 0-90 bp.

In another preferred embodiment, the length of the element Y3 is 5-30 bp, preferably 10-20 bp.

In another preferred embodiment, the length of the element Y4 is 5-30 bp, preferably 8-20 bp.

In another preferred embodiment, the length of the linker sequence is 1-30 nt.

In another preferred embodiment, part or all of the L2 and/or Y3 can be used as a barcode sequence.

In another preferred embodiment, the L2 and/or Y3 contains a barcode sequence.

In another preferred embodiment, the L2 is a barcode sequence.

In another preferred embodiment, the Y3 is a universal primer binding region.

In another preferred embodiment, the reverse primer includes a specific primer, universal primer, and barcode primer.

In another preferred embodiment, the reverse primer is used as a universal primer or a barcode primer.

In another preferred embodiment, the component (d) corresponds to the RNA polymerase initiation region in the component (a).

In another preferred embodiment, the sgRNA synthesis system further includes one or more components selected from the group consisting of:

(e) DTT;

(f) spermidine;

(g) glycerin;

(h) RNase Free water;

(i) Triton-X100;

(j) RNase inhibitor;

(k) ammonium sulfate;

(l) Tween 20

(m) substrate for RNA synthesis;

(n) substrate for DNA synthesis;

(o) magnesium ion;

(p) buffer.

In another preferred embodiment, the substrate for RNA synthesis includes: nucleoside monophosphate, nucleoside triphosphate, and a combination thereof.

In another preferred embodiment, the substrate for DNA synthesis includes: deoxynucleoside monophosphate, deoxynucleoside triphosphate, and a combination thereof.

In another preferred embodiment, the DNA polymerase is selected from the group consisting of Klenow polymerase, Taq enzyme, Pfu enzyme, KOF enzyme, Bst enzyme, Phi29 enzyme, and a combination thereof.

In another preferred embodiment, the RNA polymerase is selected from the group consisting of T7 RNA polymerase, Sp6 RNA polymerase, U6 RNA polymerase, T3 RNA polymerase, and a combination thereof.

In another preferred embodiment, the magnesium ion is derived from a magnesium ion source, and the magnesium ion source is selected from the group consisting of magnesium chloride, magnesium acetate, magnesium glutamate, magnesium phosphate, and a combination thereof.

In another preferred embodiment, the buffer is selected from the group consisting of Tris-HCl, 4-hydroxyethylpiperazine ethane sulfonic acid, tris(hydroxymethyl) aminomethane, phosphate buffer, citric acid buffer, and a combination thereof.

In another preferred embodiment, in the sgRNA synthesis system, the mass concentration (wt %) of the component (a) is 0.0008%-0.006%, preferably, 0.001%-0.005%, more preferably, 0.0013%-0.00438%, based on the total weight of the sgRNA synthesis system.

In another preferred embodiment, in the sgRNA synthesis system, the molar concentration (mol/L) of the component (a) is 0.3 umol/L-2 umol/L, preferably 0.5 umol/L-1.5 umol/L, more preferably, 0.8 umol/L-1.2 umol/L, based on the total volume of the sgRNA synthesis system.

In another preferred embodiment, in the sgRNA synthesis system, the mass concentration (wt %) of the component (b) is 0.00024%-0.0011%, preferably, 0.0003%-0.00093%, more preferably, 0.00039%-0.00081%, based on the total weight of the sgRNA synthesis system.

In another preferred embodiment, in the sgRNA synthesis system, the enzyme activity (U/uL) of the component (c) is 0.05 U/uL-0.4 U/uL, preferably 0.08 U/uL-0.25 U/uL, more preferably, 0.1 U/uL-0.2 U/uL, based on the total volume of the sgRNA synthesis system.

In another preferred embodiment, in the sgRNA synthesis system, the enzyme activity (U/uL) of the component (d) is 1 U/uL-6 U/uL, preferably, 2 U/uL-5 U/uL, more preferably, 3 U/uL-4 U/uL, based on the total volume of the sgRNA synthesis system.

In another preferred embodiment, in the sgRNA synthesis system, the molar concentration (mol/L) of the component (e) is 0.1 mmol/L-3 mmol/L, preferably, 0.3 mmol/L-2 mmol/L, more preferably, 0.5 mmol/L-1.5 mmol/L, based on the total volume of the sgRNA synthesis system.

In another preferred embodiment, in the sgRNA synthesis system, the molar concentration (mol/L) of the component (f) is 0.8 mmol/L-4 mmol/L, preferably 1.2 mmol/L-3 mmol/L, more preferably, 1.5 mmol/L-2.5 mmol/L, based on the total weight of the sgRNA synthesis system.

In another preferred embodiment, in the sgRNA synthesis system, the volume concentration (v/v) of the component (g) is 15%-35%, preferably, 18%-32%, more preferably, 20%-30%, based on the total volume of the sgRNA synthesis system.

In another preferred embodiment, in the sgRNA synthesis system, the volume concentration (v/v) of the component (j) is 5%-15%, preferably, 6%-13%, more preferably, 8%-11%, based on the total volume of the sgRNA synthesis system.

In another preferred example, in the sgRNA synthesis system, the volume concentration (v/v) of the component (1) is 0.2%-0.7%, preferably, 0.3%-0.6%, more preferably, 0.4%-0.6%, based on the total weight of the sgRNA synthesis system.

In another preferred embodiment, in the sgRNA synthesis system, the molar concentration (mol/L) of the component (m) is 1 mmol/L-1.5 mmol/L, preferably 1.1 mmol/L-1.4 mmol/L, more preferably, 1.2 mmol/L-1.3 mmol/L, based on the total weight of the sgRNA synthesis system.

In another preferred embodiment, in the sgRNA synthesis system, the molar concentration (mol/L) of the component (n) is 0.3 mmol/L-0.7 mmol/L, preferably 0.35 mmol/L-0.6 mmol/L, more preferably, 0.4 mmol/L-0.5 mmol/L, based on the total weight of the sgRNA synthesis system.

In another preferred embodiment, the component (m)/component (n) is 10:1-4:1, preferably, 8:1-3:1, more preferably, 5:1-2:1.

In another preferred embodiment, in the sgRNA synthesis system, the molar concentration (mol/L) of the component (o) is 3.5 mmol/L-7 mmol/L, preferably, 4 mmol/L-6.5 mmol/L, more preferably, 5 mmol/L-6 mmol/L, based on the total weight of the sgRNA synthesis system.

In another preferred embodiment, the sgRNA synthesis system has the following properties:
in the synthesis system (5 ul-200 ul, preferably 10-100 ul), the total sgRNA synthesis is ≥1 ug (1-10 ug, preferably 1-5 ug).

In a second aspect of the present invention, it provides a method for synthesizing sgRNA in vitro, comprising the steps:
(i) providing the sgRNA synthesis system according to the first aspect of the present invention;
(ii) under suitable conditions, incubating the synthesis system of step (i) for a period of time T1, thereby synthesizing the sgRNA.

In another preferred embodiment, the method further includes: (iii) optionally separating or detecting the sgRNA from the sgRNA synthesis system.

In another preferred embodiment, in the step (ii), the reaction temperature is 25° C.-42° C., preferably, 35° C.-40° C.

In another preferred embodiment, in the step (ii), the reaction time T1 is 0.5 h-8 h, preferably 0.5-3 h, preferably 0.8 h-1 h.

In a third aspect of the present invention, it provides a kit for sgRNA synthesis, comprising:

(k1) a first container, and a nucleic acid construct located in the first container, the nucleic acid construct having a 5′-3′ structure as shown in Formula I:

Y1-L1-Y2-L2-Y3-Y4 (I)

wherein

Y1 is a RNA polymerase initiation region;

L1 is none or a linker sequence;

Y2 is a target DNA sequence;

L2 is none or a linker sequence;

Y3 is a reverse primer binding region;

Y4 is none or a nucleotide sequence;

and, each “-” is independently a bond or a nucleotide linker sequence;

(k2) a second container, and a reverse primer located in the second container; and

(kt) a label or instructions.

In another preferred embodiment, the first container and the second container are the same container or different containers.

In another preferred embodiment, the kit further includes one or more containers optionally selected from the group consisting of:

(k3) a third container, and a DNA polymerase located in the third container;

(k4) a fourth container, and a RNA polymerase located in the fourth container;

(k5) a fifth container, and a substrate for RNA synthesis located in the fifth container;

(k6) a sixth container, and a substrate for DNA synthesis located in the sixth container;

(k7) a seventh container, and a magnesium ion located in the seventh container;

(k8) an eighth container, and a buffer located in the eighth container.

In a fourth aspect of the present invention, it provides a nucleic acid construct having a 5′-3′ structure as shown in Formula I:

Y1-L1-Y2-L2-Y3-Y4 (I)

wherein

Y1 is a RNA polymerase initiation region;

L1 is none or a linker sequence;

Y2 is a target DNA sequence;

L2 is none or a linker sequence;

Y3 is a reverse primer binding region;

Y4 is none or a nucleotide sequence;

and, each “-” is independently a bond or a nucleotide linker sequence.

In another preferred embodiment, the RNA polymerase is selected from the group consisting of T7 RNA polymerase, Sp6 RNA polymerase, U6 RNA polymerase, T3 RNA polymerase, and a combination thereof.

In another preferred embodiment, the RNA polymerase initiation region is a T7 RNA polymerase initiation region.

In another preferred embodiment, the sequence structure of the element Y1 is: N_{X}-TAATACGACTCACTATA (positions 2-18 of SEQ ID NO.:1)-G_{Y}, wherein N is A, T, C or G, and X is an integer of 1-6, Y is an integer of 0-2.

In another preferred embodiment, the sequence of the element Y1 is shown in SEQ ID NO.:1.

In another preferred embodiment, the sequence of the element Y1 is shown in SEQ ID NO.:2.

In another preferred embodiment, the sequence of the element Y1 is shown in SEQ ID NO.:3.

In another preferred embodiment, the length of the element Y1 is 17-40 bp, preferably, 18-25 bp.

In another preferred embodiment, the length of the element Y2 is 0-100 bp, preferably, 0-90 bp.

In another preferred embodiment, the length of the element Y3 is 5-30 bp, preferably, 10-20 bp.

In another preferred embodiment, the length of the element Y4 is 5-30 bp, preferably, 8-20 bp.

In another preferred embodiment, the length of the linker sequence is 1-30 nt.

In another preferred embodiment, the part or all of the L2 and/or Y3 can be used as a barcode sequence.

In another preferred embodiment, the L2 and/or Y3 contains a barcode sequence.

In another preferred embodiment, the L2 is a barcode sequence.

In another preferred embodiment, the Y3 is a universal primer binding region.

It should be understood that, within the scope of the present invention, the technical features specifically described above and below (such as the Examples) can be combined with each other, thereby constituting a new or preferred technical solution which needs not be described one by one.

### DESCRIPTION OF DRAWINGS

FIG. 1 shows an electrophoresis of sgRNA synthesized by in vitro transcription in schemes A and B. A and B represent the experimental systems of schemes A and B, respectively; lane 1 is Marker; lanes 2-5 are 0.5 h, 1 h, 1.5 h, 2 h of reaction time, respectively.

FIG. 2 shows an electrophoresis of sgRNA synthesized by in vitro transcription in schemes C and D. C and D represent the experimental systems of schemes C and D, respectively; lane 1 is Marker; lanes 2-5 are respectively 0.5 h, 1 h, 1.5 h, 2 h of reaction time.

FIG. 3 shows an electrophoresis of sgRNA synthesized by in vitro transcription in scheme E. Lane 5 is Marker; lanes 1-4 are 0.5 h, 1 h, 1.5 h, 2 h of reaction time, respectively.

FIG. 4 shows a comparison electrophoresis diagram of sgRNA in vitro transcription between schemes F and C. Lane 2 is DNA Marker 2000; Lane 1 is scheme F; Lane 3 is scheme C.

FIG. 5 shows the positive representation of the positive control sgRNA added to the CRISPR/Cas cutting system; MK is DNA Ladder 2000; A1, A2, A3, and A4 represent the sgRNA obtained by adding the four reaction time in the scheme A to the CRISPR/Cas cutting system, respectively, that is, 0.5 h, 1 h, 1.5 h, 2 h; B1-4, C1-4, D1-4 and so on are the sgRNA obtained by adding the four reaction time of schemes B, C, and D; F1, F2 are scheme F, the sgRNA amplification time is 3 h and 2 h, respectively.

### DETAILED DESCRIPTION OF INVENTION

After extensive and intensive research, through a large number of screening and exploration, a simple, convenient, time-saving, efficient and economical sgRNA synthesis method in vitro has been discovered unexpectedly for the first time. Specifically, the sgRNA synthesis system of the present invention can significantly increase the synthesis yield of sgRNA, the yield is ≥1 ug (1-10 ug, preferably 1-5 ug), and the entire process time can be greatly shortened (for example, reduced to less than 1 hour), the steps are simple, and the average cost is also greatly reduced (down by about 50%). On this basis, the present inventor has completed the present invention.

Constructs of the Present Invention

The present invention provides a nucleic acid construct by combining a RNA polymerase initiation region (such as T7 RNA polymerase initiation region), target DNA sequence, reverse primer binding region (such as specific primer, universal primer, barcode primer or a reverse primer can be used as a universal primer, a barcode primer) and an optional additional nucleotide sequence, which are connected by a linker sequence, so that sgRNA can be synthesized in vitro simply, conveniently, time-saving, and efficiently, and the cost is very low. The construct of the present invention is as described in the first aspect of the present invention.

The various elements used in the construct of the present invention are known in the art, so that those skilled in the art can use conventional methods, such as PCR methods, artificial chemical synthesis methods, and enzyme digestion methods to obtain corresponding elements, and then connecting them together by the well-known DNA ligation technique to form the construct of the present invention.

In the present invention, the sequence structure of the RNA polymerase initiation region is: N_{X}-TAATACGACTCACTATA (positions 2-18 of SEQ ID NO.:1)-G_{Y}, wherein N is A, T, C or G, and X is an integer of 1-6, and Y is an integer of 0-2.

In a preferred embodiment, the RNA polymerase initiation region is a T7 polymerase initiation region, and the sequence is shown in SEQ ID NO.:1.

In a preferred embodiment, the RNA polymerase initiation region is an SP6 polymerase initiation region, and the sequence is shown in SEQ ID NO.:2.

In a preferred embodiment, the RNA polymerase initiation region is a T3 polymerase initiation region, and the sequence is shown in SEQ ID NO.:3.

sgRNA Synthesis System

The present invention provides an in vitro sgRNA synthesis system, comprising:

(a) the nucleic acid construct according to the first aspect of the present invention;

(b) reverse primer;

(c) DNA polymerase; and

(d) RNA polymerase.

In a preferred embodiment, the in vitro sgRNA synthesis system further includes:

(e) DTT;

(f) Spermidine;

(g) Glycerin;

(h) RNase Free water;

(i) Triton-X100;

(j) RNase inhibitor;

(k) Ammonium sulfate;

(l) Tween 20

(m) Substrate for RNA synthesis;

(n) Substrate for DNA synthesis;

(o) Magnesium ion;

(p) Buffer.

In the present invention, the reverse primer is not particularly limited, and may include a universal primer or a barcode primer, or may be used as a universal primer or a barcode primer. Usually, the concentration of the reverse primer is 1 uM.

In the present invention, the RNA polymerase is not particularly limited, and can be selected from one or more RNA polymerases. A typical RNA polymerase is T7 RNA polymerase. Generally, the concentration of RNA polymerase is 3.5 U/ul.

In the present invention, the DNA polymerase is not particularly limited, and can be selected from one or more DNA polymerases, including (but not limited to): Klenow polymerase, Taq enzyme, Pfu enzyme, KOF enzyme, Bst enzyme, and/or Phi29 enzyme. A typical DNA polymerase is Taq enzyme. Generally, the concentration of DNA polymerase is 0.15 U/ul.

In the present invention, the nucleoside triphosphate mixture in the sgRNA synthesis system is adenosine triphosphate, guanosine triphosphate, cytosine triphosphate and uridine triphosphate. In the present invention, the concentration of various mononucleotides is not particularly limited. Generally, the concentration of each mononucleotide is 0.1 mM-0.5 mM, preferably 0.25 mM-0.35 mM.

In the present invention, the deoxynucleoside triphosphate mixture in the sgRNA synthesis system is adenine deoxynucleoside triphosphate, guanine deoxynucleoside triphosphate, cytosine deoxynucleoside triphosphate and thymine deoxyribonucleoside triphosphate. In the present invention, the concentration of various monodeoxynucleotides is not particularly limited. Generally, the concentration of each monodeoxynucleotide is 0.05 mM-0.15 mM, preferably 0.1 mM-0.13 mM.

In the present invention, the concentration of the components (e)-(p) is not particularly limited, and commonly used concentrations of components (e)-(p) can be used to synthesize sgRNA.

In a particularly preferred embodiment, the sgRNA synthesis system includes:

| | | |
|---|---|---|
| Tris-HCl [symbol] | 40 | mM [symbol] |
| MgCl₂ [symbol] | 5-6 | mM [symbol] |
| DTT [symbol] | 0.8-1 | mM [symbol] |
| spermidine | 1.8-2.5 | mM [symbol] |
| component A | 0.5-1 | μM [symbol] |
| Component B | 0.5-1 | μM [symbol] |
| dNTPs [symbol] | 0.5-1 | mM [symbol] |
| NTPs [symbol] | 1.25-1.5 | mM [symbol] |
| T7 RNA polymerase | 3.5-5 | U/ul [symbol] |
| Taq DNA polymerase | 0.15-0.2 | U/ul [symbol] |

Kit

The present invention provides a kit for sgRNA synthesis, comprising:

(k1) a first container, and the nucleic acid construct according to the first aspect of the present invention located in the first container;

(k2) a second container, and the reverse primer located in the second container; and

(kt) a label or instructions.

In a preferred embodiment, the first container and the second container are the same container or different containers.

A particularly preferred kit for in vitro sgRNA synthesis includes an in vitro sgRNA synthesis system, which includes: Tris-HCl 40 mM, MgCl₂ 6 mM, DTT 1 mM, spermidine 2 mM, component A 1 uM, component B 1 uM, dNTPs 0.5 mM, NTPs 1.25 mM, T7 RNA polymerase 3.5 U/ul, Taq DNA polymerase 0.15 U/ul.

The main advantages of the present invention include:

(1) The present invention develops for the first time a simple, convenient, time-saving, efficient and economical sgRNA synthesis method in vitro.

(2) The method of the present invention can greatly reduce the entire process time (such as, it is reduced to less than 1 hour), the steps are simple and the average cost is also greatly reduced (down by about 50%).

(3) The method of the present invention eliminates the PCR amplification and gel recovery steps of the traditional RNA in vitro transcription method, and reduces the duration of the entire process from 4 to 6 hours to 1 hour. The present invention also reduces the reagents that need to be added, making the entire operation more convenient.

(4) After eliminating the need for PCR amplification, the method of the present invention generates RNA in an amount far exceeding that of traditional transcription methods. Under the condition of reducing the total time, the output of the one-step method also exceeds that of the traditional RNA in vitro transcription method, and the output is ≥1 ug (1-10 ug, preferably 1-5 ug).

The present invention will be further explained below in conjunction with specific embodiments. It should be understood that these embodiments are only used to illustrate the present invention and not to limit the scope of the present invention. The experimental methods without specific conditions in the following examples usually follow the conventional conditions, such as the conditions described in Sambrook et al., Molecular Cloning: Laboratory Manual (New York: Cold Spring Harbor Laboratory Press, 1989), or according to manufacturing The conditions suggested by the manufacturer. Unless otherwise specified, percentages and parts are percentages by weight and parts by weight.

Unless otherwise specified, the materials and reagents used in the embodiments of the present invention are all commercially available products.

General Method

In the present invention, the reaction buffer for sgRNA in vitro one-step transcription synthesis reaction has specific components: Tris-HCl, MgCl₂, DTT, spermidine, sgRNA-R, dNTPs, NTP.

Preferably, Buffer is specifically:

| reaction buffer | final concentration | |
|---|---|---|
| Tris-HCl [symbol] | 40 | mM [symbol] |
| MgCl₂ [symbol] | 6 | mM [symbol] |
| DTT [symbol] | 1 | mM [symbol] |
| spermidine | 2 | mM [symbol] |
| sgRNA-R [symbol] | 1 | uM [symbol] |
| dNTPs [symbol] | 1.25 | mM [symbol] |
| NTP [symbol] | 0.5 | mM [symbol] |

In the present invention, there is also provided an Enzyme Mix for in vitro one-step transcription synthesis reaction of sgRNA. Mix is a mixture of two enzymes, one is DNA polymerase (one of Klenow polymerase, Taq enzyme, Pfu enzyme, KOF enzyme, Bst enzyme, and Phi29 enzyme), the other is RNA polymerase (one of T₇ enzyme, Sp6 enzyme, U6 enzyme).

Preferably, Enzyme Mix is a combination of Taq enzyme and T₇ RNA polymerase.

The implementation steps of the present invention are:

I. Primer Design of sgRNA Template

1. sgRNA Forward (F) Primer Design

The 5′ end 20 bp of the Target DNA sequence PAM (NGG) is selected for forward (F) primer design. The primer structure includes T7 promoter (20 bp), Target DNA fragment (20 bp) and the fragment actually combined with the reverse primer (10-25 bp).

The schematic diagram of primer design is as follows:

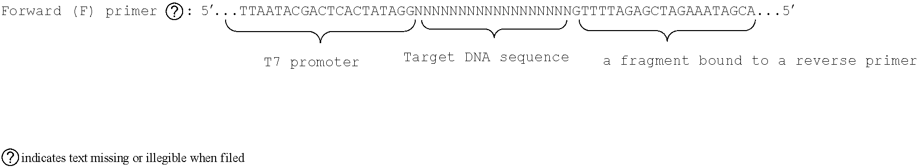

For example, assuming that the Target DNA fragment is

(SEQ ID NO.: 4)
5′-TCCCCGCTGCAGCATAGTGAGCCCAGAANGGCATT-3′,

the schematic diagram of primer design is as follows:

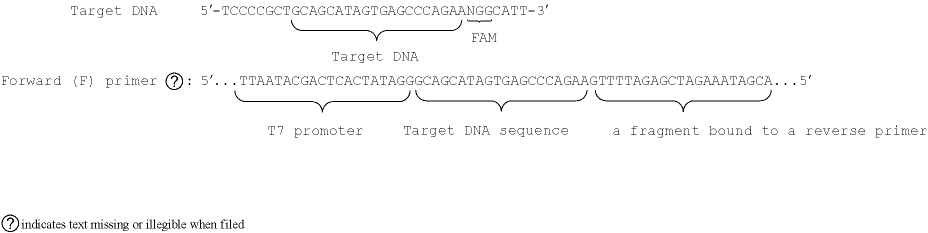

II. sgRNA In Vitro Transcription System

Designing and synthesizing forward primers according to the primer design requirements, prepared according to the following system, and reacted at 37° C. for 1 hour:

| transcription system | Dosage (μL) |
|---|---|
| reaction buffer | 10 [symbol] |
| Forward primer F(10 μM) | 2 [symbol] |
| Enzyme Mix [symbol] | 2 [symbol] |
| RNase Free Waters [symbol] | To 40 [symbol] |

III. Purification of sgRNA

3.1 Removal of Template DNA:

Adding 1 μl DNase I to the transcription product, reacted at 37° C. for 10 minutes to remove the DNA template, the reaction product was put at 75° C. and reacted for 10 minutes, and the resulting sgRNA was stored at −20° C.

3.2 sgRNA Purification (Optional):

1. Making up 20 μl of transcription system to 300 μl with RNase Free Water, adding an equal volume of phenol, chloroform, and isoamyl alcohol (25:24:1), mixed thoroughly, and centrifuged at 12000 rpm at 4° C. for 15 min.

2. Taking the supernatant, adding 300 μl chloroform, mixed well, centrifuged at 12000 rpm, 4° C. for 5 min.

3. Taking about 200 μl of the supernatant. In order to increase the recovery volume, adding 1001 of RNase Free Water to the lower layer, mixed well, centrifuged at 4° C. for 5 minutes, and taking the supernatant.

4. Combining the supernatants taken twice to a total volume of about 300-350 μl, add 2 volumes of isopropanol and 1/10 volume of 3M NaAc prepared with RNase Free Water, and precipitated overnight at −80° C.

5. Centrifuging it to get the precipitate, 1 ml of 75% ethanol prepared with RNase Free Water was added, and washed twice.

6. Dissolving RNA with 20 μl RNase Free Water, checking the quality by electrophoresis and determining the concentration.

### Example 1 Designing Five In Vitro Transcription Systems A, B, C, D, and E, and Designing F as a Traditional In Vitro Transcription System to Compare the Advantages and Disadvantages of the Schemes

Designing sgRNA forward primer (sgRNA-F):
(SEQ ID NO.: 5)
5′-TTAATACGACTCACTATAGGGCAGCATAGTGAGCCCAGAAGTTTTAG
AGCTAGAAATAGCA-3′
Designing sgRNA reverse primer (sgRNA-R):
(SEQ ID NO.: 6)
5′-TGCTATTTCTAGCTCTAAAAC-3′

Dissolving sgRNA-F and sgRNA-R in RNase Free Water to prepare a concentration of 5 uM.

1. In the experimental system of scheme A and B, all the components except the primers were mixed together to prepare reaction buffer 1. The components were as follows:

| reaction buffer 1 | Scheme A (μL) | Scheme B (μL) |
|---|---|---|
| Tris-HCl [symbol] | 80 [symbol] | 80 [symbol] |
| MgCl₂ [symbol] | 24 [symbol] | 24 [symbol] |
| DTT [symbol] | 2 [symbol] | 2 [symbol] |
| spermidine | 40 [symbol] | 40 [symbol] |
| Taq DNA polymerase | 100 [symbol] | 100 [symbol] |
| dNTPs (10 mM) [symbol] | 100 [symbol] | 100 [symbol] |
| NTP (25 mM) [symbol] | 100 [symbol] | 200 [symbol] |
| T7 RNA polymerase | 100 [symbol] | 200 [symbol] |
| Glycerin (stock solution) | 150 [symbol] | 150 [symbol] |
| Anhydrous RNAase (RNase | 304 [symbol] | 104 [symbol] |
| Free Water) [symbol] | | |
| total volume | 1000 [symbol] | 1000 [symbol] |

20 μL reaction system was prepared as follows for schemes A and B:

Total reaction volume 20 μL

Reaction buffer 1 10 μL

sgRNA-F 2 μL

sgRNA-R 2 μL

RNase Free Water 6 μL

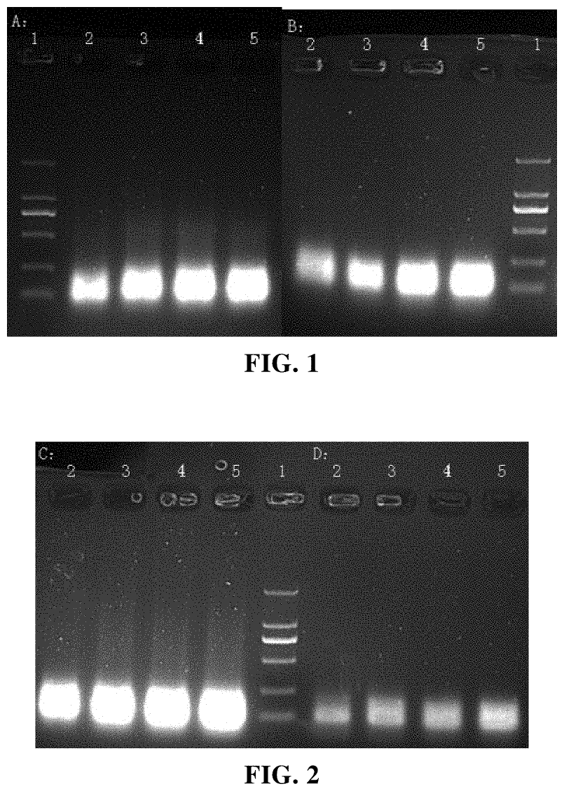

Using the schemes A and B to set 4 repetitions respectively, placing them in a PCR machine, and reacted at 37° C. The reaction time of 4 repetitions was 0.5 h, 1 h, 1.5 h and 2 h, respectively. After the reaction, 1 μl DNaseI was added and reacted at 37° C. for 10 minutes to remove the DNA template, and the product was subjected to electrophoresis. The result is shown in FIG. 1.

The results show that with the reaction time from 0.5 h to 2 h, the yield is increased; the yield of the scheme A is higher than that of the scheme B, wherein the yield of the scheme A is 1.2 ug (2 h), and the yield of the scheme B is 1 ug (2 h).

2. In the experimental system of schemes C and D, mixing other components except the forward primer and two enzymes to prepare reaction buffer 2. The components were as follows:

| reaction buffer 2 | Scheme C (μL) | Scheme D (μL) |
|---|---|---|
| Tris-HCl [symbol] | 80 [symbol] | 80 [symbol] |
| MgCl₂ [symbol] | 24 [symbol] | 24 [symbol] |
| DTT [symbol] | 2 [symbol] | 2 [symbol] |
| spermidine | 40 [symbol] | 40 [symbol] |
| sgRNA-R [symbol] | 200 [symbol] | 200 [symbol] |
| dNTPs (10 mM) [symbol] | 100 [symbol] | 100 [symbol] |
| NTP (25 mM) [symbol] | 100 [symbol] | 200 [symbol] |
| RNase Free Water [symbol] | 454 [symbol] | 354 [symbol] |
| total volume | 1000 [symbol] | 1000 [symbol] |

Mixing Taq enzyme and T7 RNA enzyme together to prepare Enzyme Mix:

| Enzyme Mix | Volume (μL) |
|---|---|
| Taq DNA polymerase | 6 [symbol] |
| T7 RNA Polymerase | 14 [symbol] |
| total volume | 20 [symbol] |

20 μL reaction system was prepared as follows for Schemes C and D:

| | | |
|---|---|---|
| Total reaction volume | 20 | μL |
| Reaction buffer 2 | 10 | μL |
| sgRNA-F | 2 | μL |
| Enzyme Mix | 2 | μL |
| RNase Free Water | 6 | μL |

Using the schemes C and D to set 4 repetitions respectively, placing them in a PCR machine, and reacted at 37° C. The reaction time of 4 repetitions was 0.5 h, 1 h, 1.5 h and 2 h respectively. After the reaction, 1 μl DNaseI was added and reacted at 37° C. for 10 minutes to remove the DNA template, and the product was subjected to electrophoresis. The result is shown in FIG. 2.

The results show that with the reaction time from 0.5 h to 2 h, the yield is improved; the yield of the scheme C is much higher than that of the scheme D, in which the yield of the scheme C is 2 ug (2 h), and the yield of the scheme D is 400 ng (2 h).

3. Scheme E, Transcription Mix was prepared respectively:

| Transcription mix | Volume (μL) |
|---|---|
| Tris-HCl [symbol] | 80 [symbol] |
| MgCl₂ [symbol] | 32 [symbol] |
| DTT [symbol] | 10 [symbol] |
| spermidine | 40 [symbol] |
| NTP (25 mM) [symbol] | 80 [symbol] |
| Triton-X100 [symbol] | 10 [symbol] |
| Glycerin (stock solution) | 150 [symbol] |
| RNase inhibitor | 100 [symbol] |
| T7 RNA polymerase | 150 [symbol] |
| RNase Free Water [symbol] | 388 [symbol] |
| total volume | 1000 [symbol] |

and Template Amplification Mix:

| template amplification mix | Volume (μL) |
|---|---|
| Tris-HCl [symbol] | 375 [symbol] |
| (NH₄)₂SO₄ [symbol] | 100 [symbol] |
| MgCl₂ [symbol] | 20 [symbol] |
| Tween 20 [symbol] | 50 [symbol] |
| DTT [symbol] | 2.5 [symbol] |
| Glycerin (stock solution) | 250 [symbol] |
| HotStart Taq DNA polymerase | 80 [symbol] |
| RNase Free Water [symbol] | 122.5 [symbol] |
| total volume | 1000 [symbol] |

20 μL reaction system was prepared as follows for the scheme E:

| | | |
|---|---|---|
| Total reaction volume | 20 | μL |
| Transcription Mix | 10 | μL |
| Template Amplification Mix | 4 | μL |
| sgRNA-F | 2 | μL |
| sgRNA-R | 2 | μL |
| RNase Free Water | 2 | μL |

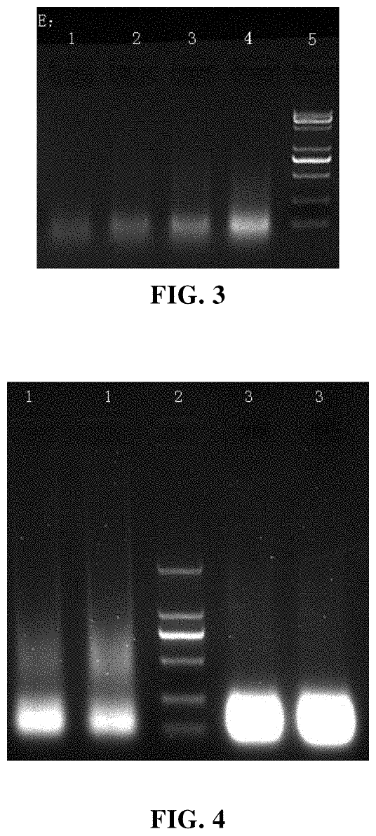

Using scheme E to do 4 repetitions, placed in a PCR machine, and reacted at 37° C. The duration of the 4 repeated reactions was 0.5 h, 1 h, 1.5 h, and 2 h respectively. After the reaction, 1 μl DNaseI was added and reacted at 37° C. for 10 minutes to remove the DNA template, and the product was electrophoresed. The result is shown in FIG. 3.

The results show that with the reaction time from 0.5 h to 2 h, the yield is improved, the yield of scheme E is very low, and the yield of scheme E is 200 ng (2 h).

1. Scheme F, Transcription Mix was prepared respectively:

| Transcription Mix | Volume (μL) |
|---|---|
| Tris-HCl [symbol] | 80 [symbol] |
| MgCl₂ [symbol] | 32 [symbol] |
| DTT [symbol] | 10 [symbol] |
| spermidine | 40 [symbol] |
| NTP (25 mM) [symbol] | 80 [symbol] |
| Triton-X100 [symbol] | 10 [symbol] |
| Glycerin (stock solution) | 150 [symbol] |
| RNase inhibitor | 100 [symbol] |
| T7 RNA polymerase | 150 [symbol] |
| RNase Free Water [symbol] | 388 [symbol] |
| total volume | 1000 [symbol] |

and Template Amplification Mix:

| Template | |
|---|---|
| Amplification Mix | Volume (μL) |
| Tris-HCl [symbol] | 375 [symbol] |
| (NH₄)₂SO₄ [symbol] | 100 [symbol] |
| MgCl₂ [symbol] | 20 [symbol] |
| Tween 20 [symbol] | 50 [symbol] |
| DTT [symbol] | 2.5 [symbol] |
| Glycerin (stock solution) | 250 [symbol] |
| HotStart Taq DNA | 80 [symbol] |
| polymerase | |
| RNase Free Water [symbol] | 122.5 [symbol] |
| total volume | 1000 [symbol] |

The forward primer, reverse primer and Template Amplification Mix were prepared into the following 20 μL PCR reaction system:

| | | |
|---|---|---|
| Total reaction volume | 20 | μL |
| Template Amplification Mix | 4 | μL |
| sgRNA-F | 2 | μL |
| sgRNA-R | 2 | μL |
| RNase Free Water | 12 | μL |

Putting the reaction system into a PCR machine and performing PCR amplification according to the following procedures:

1 95° C. 1 min

2 95° C. 20 s

3 68° C. 20 s returns to 2, 25 cycles

4 68° C. 1 min

the following 20 μL reaction system was prepared for PCR amplification products:

| | | |
|---|---|---|
| PCR product | 10 | μL |
| Transcription Mix | 10 | μL |

After reacting for 2 hours at 37° C., the result of sgRNA synthesis was compared with that of scheme C as shown in FIG. 4.

The results show that scheme C has the best effect, and its synthesized sgRNA yield is the highest, reaching 1 ug-2 ug, and unexpectedly, its yield far exceeds the traditional sgRNA synthesis method (the sgRNA yield of the traditional synthesis method is about 100 ng-200 ng), which is improved by 5-10 times.

### Example 2 Detecting the Activity Effect of sgRNA Synthesized by One-Step In Vitro Transcription in the CRISPR/Cas System

CRISPR. Cas9 in vitro cleavage reaction system:

| | | | |
|---|---|---|---|
| | total volume | 20 | μL [symbol] |
| | CRISPR reaction buffer | 2 | μL [symbol] |
| | 1.5 kb Target DNA Control | 6 | μL [symbol] |
| {open oversize brace} | Cas9 endonuclease | 2 | μL [symbol] |
| | RNase Free Water | 7 | μL [symbol] |
| | sgRNA | 3 | μL [symbol] |

19 systems were prepared. Positive control sgRNA was added to one system, and the sgRNA obtained by in vitro transcription was added to the rest. The reaction was performed at 37° C. for 1 hour, 70° C. for 10 minutes, and 10° C. for 10 minutes.

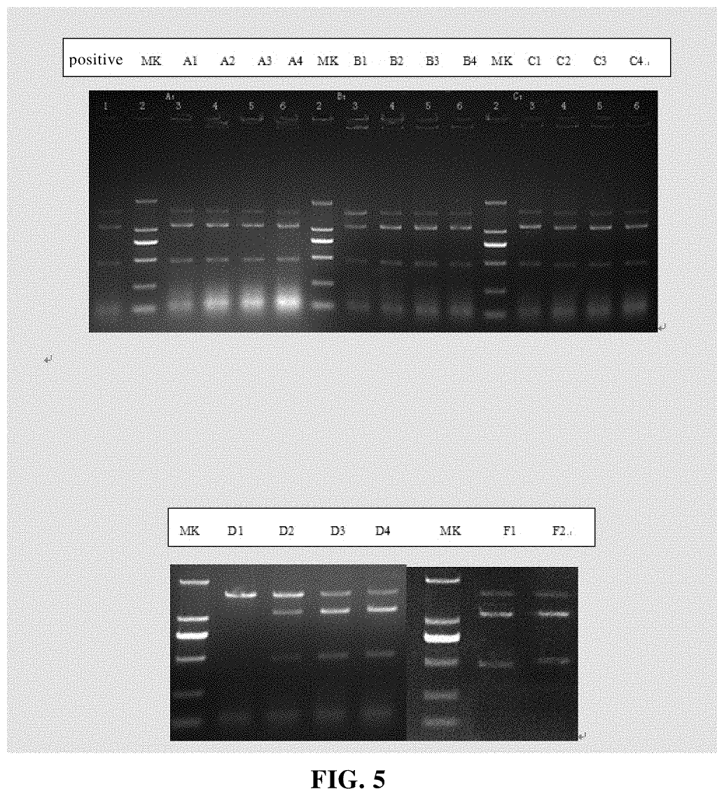

FIG. 5 shows the electrophoresis results after the reaction.

The results show that when the NTP/dNTP in the reaction system is 10:1-4:1, preferably, 8:1-3:1, more preferably, 5:1-2:1, the sgRNA synthesized by the present invention has the activity of guiding Cas9 to cut specific sites.

In addition, except for the sgRNA obtained from the 0.5 h transcription time of the scheme D, the amount is too small to guide the Cas9 protein to cleave DNA, all other DNA is cleaved by the Cas9 protein guided by the sgRNA, further showing that the sgRNA synthesized by the one-step in vitro transcription method of the present invention has the activity of guiding Cas9 to cleave specific sites.

The comparison between scheme C and scheme D:

| reaction buffer 2 | Scheme C (μL) | Scheme D (μL) |
|---|---|---|
| Tris-HCl [symbol] | 80 [symbol] | 80 [symbol] |
| MgCl₂ [symbol] | 24 [symbol] | 24 [symbol] |
| DTT [symbol] | 2 [symbol] | 2 [symbol] |
| spermidine | 40 [symbol] | 4 [symbol] |
| sgRNA-R [symbol] | 200 [symbol] | 200 [symbol] |
| dNTPs (10 mM) [symbol] | 100 [symbol] | 100 [symbol] |
| NTPs (25 mM) [symbol] | 100 [symbol] | 200 [symbol] |
| RNase Free Water [symbol] | 454 [symbol] | 354 [symbol] |
| total volume | 1000 [symbol] | 1000 [symbol] |

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A sgRNA synthesis system, comprising:
(a) a nucleic acid construct, which has a 5′-3′ structure as shown in Formula I:
Y1-L1-Y2-L2-Y3-Y4 (I)
wherein
Y1 is a RNA polymerase initiation region;
L1 is none or a linker sequence;
Y2 is a target DNA sequence;
L2 is none or a linker sequence;
Y3 is a reverse primer binding region;
Y4 is none or a nucleotide sequence;
and, each “-” is independently a bond or a nucleotide linker sequence;
(b) a reverse primer;
(c) a DNA polymerase; and
(d) a RNA polymerase.

2. The sgRNA synthesis system of claim 1, wherein the RNA polymerase is selected from the group consisting of T7 RNA polymerase, Sp6 RNA polymerase, U6 RNA polymerase, T3 RNA polymerase, and a combination thereof.

3. The sgRNA synthesis system of claim 1, wherein part or all of the L2 and/or Y3 can be used as a barcode sequence.

4. The sgRNA synthesis system of claim 1, wherein the L2 is a barcode sequence.

5. The sgRNA synthesis system of claim 1, wherein the Y3 is a universal primer binding region.

6. The sgRNA synthesis system of claim 1, wherein the sequence structure of the element Y1 is: N_{X}-TAATACGACTCACTATA (positions 2-18 of SEQ ID NO.:1)-G_{Y}, wherein N is A, T, C or G, and X is an integer of 1-6, Y is an integer of 0-2.

7. The sgRNA synthesis system of claim 1, wherein the sgRNA synthesis system further includes one or more components selected from the group consisting of:
(e) DTT;
(f) spermidine;
(g) glycerin;
(h) RNase Free water;
(i) Triton-X100;
(j) RNase inhibitor;
(k) ammonium sulfate;
(l) Tween 20
(m) substrate for RNA synthesis;
(n) substrate for DNA synthesis;
(o) magnesium ion;
(p) buffer.

8. A method for synthesizing sgRNA in vitro, comprising the steps:
(ii) providing the sgRNA synthesis system of claim 1;
(ii) under suitable conditions, incubating the synthesis system of step (i) for a period of time T1, thereby synthesizing the sgRNA.

9. The method of claim 8, wherein the method further includes: (iii) optionally separating or detecting the sgRNA from the sgRNA synthesis system.

10. A kit for sgRNA synthesis, comprising:
(k1) a first container, and a nucleic acid construct located in the first container, the nucleic acid construct having a 5′-3′ structure as shown in Formula I:
Y1-L1-Y2-L2-Y3-Y4 (I)
wherein
Y1 is a RNA polymerase initiation region;
L1 is none or a linker sequence;
Y2 is a target DNA sequence;
L2 is none or a linker sequence;
Y3 is a reverse primer binding region;
Y4 is none or a nucleotide sequence;
and, each “-” is independently a bond or a nucleotide linker sequence;
(k2) a second container, and a reverse primer located in the second container; and
(kt) a label or instructions.

11. A nucleic acid construct having a 5′-3′ structure as shown in Formula I:
Y1-L1-Y2-L2-Y3-Y4 (I)
wherein
Y1 is a RNA polymerase initiation region;
L1 is none or a linker sequence;
Y2 is a target DNA sequence;
L2 is none or a linker sequence;
Y3 is a reverse primer binding region;
Y4 is none or a nucleotide sequence;
and, each “-” is independently a bond or a nucleotide linker sequence.

12. The nucleic acid construct of claim 11, wherein the RNA polymerase initiation region is a T7 RNA polymerase initiation region.

13. The nucleic acid construct of claim 11, wherein the sequence structure of the element Y1 is: N_{X}-TAATACGACTCACTATA (positions 2-18 of SEQ ID NO.:1)-G_{Y}, wherein N is A, T, C or G, and X is an integer of 1-6, Y is an integer of 0-2.

14. The nucleic acid construct of claim 11, wherein the sequence of the element Y1 is shown in SEQ ID NO.:1.

15. The nucleic acid construct of claim 11, wherein the sequence of the element Y1 is shown in SEQ ID NO.:2.

16. The nucleic acid construct of claim 11, wherein the sequence of the element Y1 is shown in SEQ ID NO.:3.p


SEQUENCE LISTING (6 sequences)

SEQ ID NO: 1 - DNA, 20 bp, artificial sequence
  synthesized
ttaatacgactcactatagg

SEQ ID NO: 2 - DNA, 19 bp, artificial sequence
  synthesized
atttaggtgacactataga

SEQ ID NO: 3 - DNA, 19 bp, artificial sequence
  synthesized
aattaaccctcactaaagg

SEQ ID NO: 4 - DNA, 35 bp, artificial sequence
  synthesized
  misc_feature (29)..(29): n is a, c, g, or t
tccccgctgcagcatagtgagcccagaanggcatt

SEQ ID NO: 5 - DNA, 61 bp, artificial sequence
  synthesized
ttaatacgactcactatagggcagcatagtgagcccagaagttttagagctagaaatagca

SEQ ID NO: 6 - DNA, 21 bp, artificial sequence
  primer
tgctatttctagctctaaaac